(19) United States
(12) Patent Application Publication
(10) Pub. No.: US 2021/0349017 A1
(43) Pub. Date: Nov. 11, 2021

(54) SYSTEMS AND METHODS FOR DETECTION OF BIOLOGICAL AGENTS USING INFRARED SPECTROSCOPY

(71) Applicant: CACI, Inc. - Federal, Arlington, VA (US)

(72) Inventors: Mihaela DINU, Freehold, NJ (US); Darren Duane HUDSON, Berkeley Heights, NJ (US); Inuk KANG, Holmdel, NJ (US)

(21) Appl. No.: 17/245,015

(22) Filed: Apr. 30, 2021

Related U.S. Application Data
(60) Provisional application No. 63/021,921, filed on May 8, 2020.

(51) Int. Cl. G01N 21/3577; C12Q 1/04; G01N 33/483; G06N 20/00; G06N 5/04

(52) U.S. Cl. CPC G01N 21/3577; C12Q 1/04; G06N 5/04; G06N 20/00; G01N 33/483

(57) ABSTRACT

This application is generally related to methods, apparatuses and systems for rapid, sensitive detection of biological agents. One aspect is directed to a system including an infrared optical source configured to output an optical beam at a pulse repetition rate greater than or equal to 1 MHz. The system also includes a medium configured to receive the optical beam in first and second locations of the medium, where each of the first and second locations is separated by a barrier, the first location includes a solvent, and the second location includes a biological agent in the solvent. The system also includes a detector configured to receive the outputted optical beam from the medium and detect infrared spectra therefrom.

### CROSS REFERENCE TO RELATED APPLICATIONS

This instant application claims the benefit of priority of U.S. Provisional Application No. 63/021,921 filed May 8, 2020, and entitled, “Method and Apparatus for Rapid Sensitive Detection of Biological Agents Using Infrared Spectroscopy,” the content of which is incorporated by reference in its entirety herein.

### FIELD

This application is generally related to system and methods for detection of biological agents, such as for example the coronavirus causing COVID-19, using infrared (IR) spectroscopy.

### BACKGROUND

Generally, existing systems and techniques in the marketplace for detecting and accurately identifying biological agents are either unreliable or inefficient. When a worldwide pandemic such as for example COVID-19 falls upon the human race, as the case in 2020, it is crucial that accurate and fast testing systems and methods be made available. This is even more relevant in areas with high infection rates

A drawback observed in existing architectures is the high rate of false negatives and/or false positives. False negatives can lead to increased infections of third persons. Stated otherwise, the infected person is unaware of his or her actual infection. Conversely, false positives may cause additional expenses inclusive of unnecessary treatment options for the non-infected person.

Spectroscopy techniques using a black body radiation source made of a ceramic heater have traditionally been used to examine biological agents. More particularly, Fourier Transform Infrared Spectroscopy (FTIR) employing a black body radiation sources is the preferred option. While spectral coverage with a black body radiation source may range from near infrared (NIR) to long-wave infrared (LWIR), the inventors of this application have observed lack of coherence and low brightness. Ultimately, the presence of ambient blackbody emission in the infrared limits spectroscopic sensitivity since source characteristics cannot be easily distinguished from thermal background radiation. As a result, conventional architectures simply cannot obtain a sufficient amount of analytes of about 1-10 μg to make a positive chemical identification.

Separately, inefficiency is also a problem in the art. The worldwide health community currently employs a reverse transcription polymerase chain reaction (RT-PCR). RT-PCR derives its sensitivity from exponential growth of genetic materials of the target virus in multi-stage cycles of gene copying. While it may offer better sensitivity, it still take a couple hours to complete. Indeed, the testing process is manually intensive including sample preparation steps performed by highly trained professionals. Hence, the sensitivity of the technique is prone to variances and false results. Separately, with continued mutations of a virus, particularly COVID-19, existing test kits may quickly become obsolete. Accurate and efficient retrieval and identification is now more important than ever.

What is desired in the art is an improved system and technique for obtaining and detecting a sufficient amount of biological agent for accurate identification.

What is also desired in the art is a machine-learning model for identifying biological agents via captured infrared data at a detector.

### SUMMARY

The foregoing needs are met, to a great extent, by the disclosed system and method for rapid and sensitive detection of biological agents.

One aspect of the application is directed to a system including an infrared optical source configured to output an optical beam at a pulse repetition rate greater than or equal to 1 MHz. The system also includes a medium configured to receive the optical beam in first and second locations of the medium, where each of the first and second locations is separated by a barrier, the first location includes a solvent, and the second location includes a biological agent in the solvent. The system also includes a detector configured to receive the outputted optical beam from the medium and detect infrared spectra therefrom.

Another aspect of the patent application is directed to a method. The method may include a step of receiving, at a detector, an optical beam output from a medium via plural locations, where the optical beam enters the medium at a pulse repetition rate greater than or equal to 1 MHz, and travels through a first path including a solvent and a second path including the solvent and a biological agent. The method may also include a step of evaluating spectra of the optical beam. In addition, the method may include a step of determining information of a biological agent based upon the evaluated spectra. Other embodiments of this aspect include corresponding computer systems, apparatus, and computer programs recorded on one or more computer storage devices, each configured to perform the actions of the methods.

Yet even another aspect of the patent application describes a computer-readable medium including program instructions that when executed by a processor of a computing device cause the computing device to perform a set of action. The computer-readable medium may include program instructions to receive, via a spectrometer employing a Fourier transform, spectra of an optical beam originating at a source and transmitted at a pulse repetition rate greater than or equal to 1 MHz through a medium including a solvent and a biological agent. The medium also includes evaluating, via a trained machine learning model, one or more of attributes of the spectra to identify the biological agent. The medium also includes identifying the biological agent based on at least a subset of the evaluated attributes. Other embodiments of this aspect include corresponding computer systems, apparatus, and computer programs recorded on one or more computer storage devices, each configured to perform the actions of the methods.

There have thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

### BRIEF DESCRIPTION OF THE DRAWINGS

In order to facilitate a fuller understanding of the invention, reference is now made to the accompanying drawings, in which like elements are referenced with like numerals. These drawings should not be construed as limiting the invention and are intended only to be illustrative.

FIG. 1 illustrates an exemplary system including an IR laser source, medium including a biological agent, and a detector capturing according to an aspect of the application.

FIG. 2 illustrates an exemplary embodiment of the medium shown in FIG. 1 including an optical beam entering and exiting the medium according to an aspect of the application.

FIG. 3 illustrates another exemplary embodiment of the medium shown as a hollow core fiber (HCF) including an optical beam entering and exiting the HCF according to an aspect of the application.

FIG. 4 illustrates an exemplary embodiment where a laser source emits optical beam that is sent to 2 HCF mediums according to an aspect of the application.

FIG. 5 illustrates a block diagram of an example computing system according to an aspect of the application.

FIG. 6 illustrates a flow chart describing a method of the present application

FIG. 7 illustrates a flow chart of program instructions stored on a computer readable medium executable by a processor of a computing device causing the computing device to perform identification of a biological agent according to an aspect of the application.

### DETAILED DESCRIPTION

In this respect, before explaining at least one embodiment of the invention in detail, it is to be understood that the invention is not limited in its application to the details of construction and to the arrangements of the components set forth in the following description or illustrated in the drawings. The invention is capable of embodiments or embodiments in addition to those described and of being practiced and carried out in various ways. Also, it is to be understood that the phraseology and terminology employed herein, as well as the abstract, are for the purpose of description and should not be regarded as limiting.

Reference in this application to “one embodiment,” “an embodiment,” “one or more embodiments,” or the like means that a particular feature, structure, or characteristic described in connection with the embodiment is included in at least one embodiment of the disclosure. The appearances of, for example, the phrases “an embodiment” in various places in the specification are not necessarily all referring to the same embodiment, nor are separate or alternative embodiments mutually exclusive of other embodiments. Moreover, various features are described which may be exhibited by some embodiments and not by the other. Similarly, various requirements are described which may be requirements for some embodiments but not by other embodiments.

It has been determined by the inventors and described herein, methods and systems for efficient and accurate detection of biological agents, such as for example, the coronavirus causing COVID-19, employing infrared (IR) spectroscopy.

One of the many benefits of the present application is its improved sensitivity gain. The present application employs a pulsed infrared (IR) laser source. The preferred IR laser source achieves about a 10-30 dB improvement in sensitivity in comparison with a black body radiation source. The laser source exhibits high sensitivity by allowing for discrimination against the background emission and 1/f detector noise via lock-in detection. Moreover, when the pulsed IR source is used in combination with a reference path and a detection path interferometric cancellation can be employed for enhanced sensitivity. It is envisaged the rejection of the common mode is about 10-20 dB. In a preferred embodiment, the rejection of the common mode is 15-20 dB. In an even more preferred embodiment, the rejection of the common mode is about 20 dB. A direct result of the present application is the ability to capture weak signals of a biological agent, such as a virus.

Another one of the many benefits of the present application is the ability to identify target species in the presence of clutters. Biological agents, and in particular, COVID-19, is characterized by a large number of polypeptide spikes and strong infrared absorption signatures around 1600-1700 cm⁻¹. The present application is capable of obtaining sufficient analytes and detecting the polypeptide spikes at the particular range to accurately provide an assessment. Moreover, the system is capable of providing the results in real-time in less than an hour. Preferably the time may be less than 5 minutes. In one or more embodiments, spectral detection is analyzed using machine learning models to rapidly provide results to clinicians in the field, or in a lab setting.

Generally, the inventors have observed spectroscopic techniques are faster than other techniques which employ biochemical assays for detecting biological agents. Namely, infrared absorption spectral analysis identifies chemical and biological samples by analyzing their spectral “fingerprint.” In other words, chemical and biological molecules exhibit distinct and sharp absorption features in the infrared region determined by constituent molecules and their structure.

Infrared chemical/biological detection is also label-free. That is, chemical and biological detection does not require chemical and biological agents. Sample preparation specific to each target species also is not required. These detection techniques are desirable for developing effective screening tools for biological agents such as viruses.

In one or more embodiments of the present application, the overall sensitivity of the system is improved by up to 50 dB in comparison with black body radiation spectroscopic techniques.

In one or more embodiments, the system for detecting and identifying a biological agent, such as for example a virus, is performed under an hour. Preferably less than 30 minutes. And even more preferably in less than 15 minutes. The exemplary embodiments of this application are at least ten times, and possibly >100 times faster than conventional techniques employing RT-PCR.

One aspect of the application describes a system including an infrared optical source configured to output an optical beam at a pulse repetition rate greater than or equal to 1 MHz. It is envisaged the pulse repetition rate may be greater than or equal to 100 MHz. The system also includes a medium configured to receive the optical beam in first and second locations of the medium, where each of the first and second locations is separated by a barrier, the first location incudes a solvent, and the second location includes a biological agent in the solvent. The system also includes a detector configured to receive the outputted optical beam from the medium and detect infrared spectra therefrom. Other embodiments of this aspect include corresponding computer systems, apparatus, and computer programs recorded on one or more computer storage devices, each configured to perform the actions of the methods.

Implementations of this aspect may include one or more of the following features. The system may include a means for interferometrically subtracting a common mode of the received optical beam prior to the detection of infrared spectra. A rejection of the common mode is greater than about 20 dB. The pulse repetition rate is greater than or equal to 1 MHz. It is envisaged the pulse repetition rate may be greater than or equal to 100 MHz. The pulse repetition rate is less than about 500 MHz. The optical beam is temporally coherent. The optical beam is spatially coherent. The medium may include a crystalline substrate and a metal film layer of about 10 to 100 nm thickness formed on the crystalline substrate. The medium includes plural hollow core fibers.

Alternatively, the pulse repetition rate is greater than or equal to 10 GHz. The system exhibits a signal to noise ratio improvement up to about 50 dB in comparison with a complementary system employing a black body radiation source instead of the infrared optical source. Implementations of the described techniques may include hardware, a method or process, or computer software on a computer-accessible medium.

A second aspect of the patent application is directed to a method. The method may include a step of receiving, at a detector, an optical beam output from a medium via plural locations, where the optical beam enters the medium at a pulse repetition rate greater than or equal to 1 MHz, and travels through a first path including a solvent and a second path including the solvent and a biological agent. It is envisaged the pulse repetition rate may be greater than 100 MHz. The pulse repetition rate may be less than 500 MHz. The method may also include a step of evaluating spectra of the optical beam. In addition, the method may include a step of determining information of a biological agent based upon the evaluated spectra. Other embodiments of this aspect include corresponding computer systems, apparatuses, and computer programs recorded on one or more computer storage devices, each configured to perform the actions of the system.

Implementations of this aspect may include one or more of the following features. The method may include a step of interferometrically subtracting a common mode of the received optical beam prior to the evaluation of spectra. A rejection of the common mode is greater than about 20 dB. The method may include a step of detecting about 1-10 picograms of analytes from the biological agent. Implementations of the described techniques may include hardware, a method or process, or computer software on a computer-accessible medium.

Yet another aspect of the patent application describes a computer readable medium including program instructions that when executed by a processor of a computing device cause the computing device to perform a set of action. The computer readable medium may include program instructions to receive, via a spectrometer employing a Fourier transform, spectra of an optical beam originating at a source and transmitted at a pulse repetition rate greater than or equal to 1 MHz through a medium including a solvent and a biological agent. It is envisaged the pulse repetition rate may be greater than 100 MHz. The pulse repetition rate may be less than 500 MHz The medium also includes evaluating, via a trained machine learning model, one or more of attributes of the spectra to identify the biological agent. The medium also includes identifying the biological agent based on at least a subset of the evaluated attributes. Other embodiments of this aspect include corresponding computer systems, apparatuses, and computer programs recorded on one or more computer storage devices, each configured to perform the actions of the methods.

Implementations may include one or more of the following features. The computer readable data provides spectral identification information in the form of analyte specific peaks, troughs, and other signatures. This information uniquely identifies various analytes much in the way a fingerprint is used to uniquely identify a human. By employing pattern recognition techniques, including machine-learning, the present application will rapidly identify analytes in the presence of clutterers and background noise. The evaluation includes assigning each of the attributes a score based upon a likelihood of similarity with at least one respective attribute in training data used by the machine learning model. The program instructions when executed by the processor further causes the computing device to transmit the identified biological agent over a network to another computing device. Interferometric subtraction of a common mode of the optical beam occurs prior to receiving the spectra. A rejection of the common mode is greater than about 20 dB. About 1-10 picograms of analytes of the biological agent are captured from the optical beam to produce the spectra. The pulse repetition rate is less than about 500 MHz. Implementations of the described techniques may include hardware, a method or process.

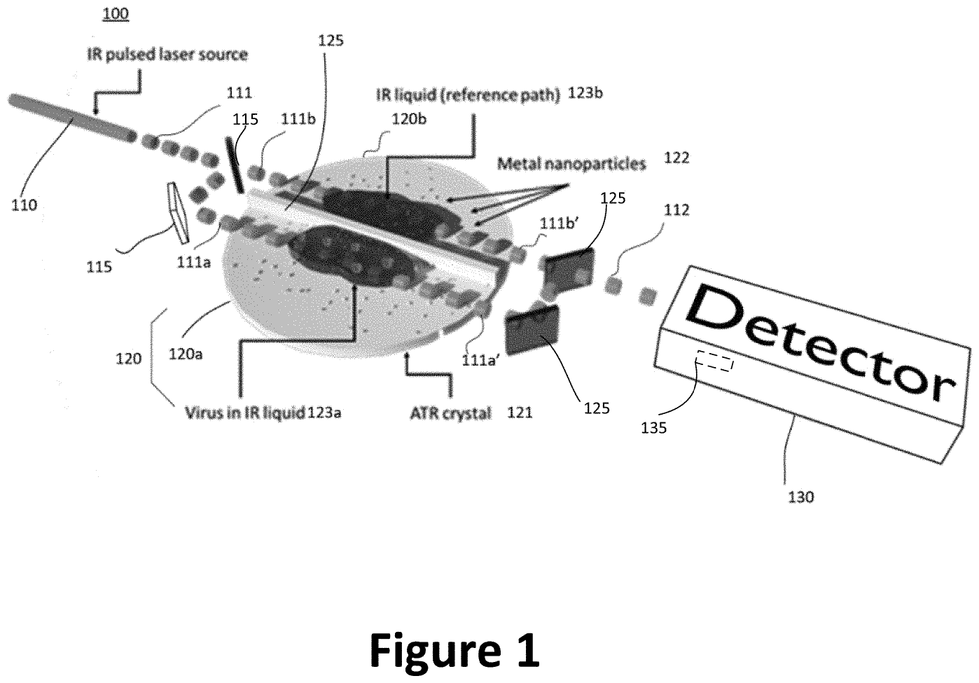

FIG. 1 depicts an exemplary system 100 for detecting and identifying biological agents. The system 100 includes a laser source 110, a medium 120 including an unknown biological agent, and a detector 130. It is envisaged that the instant technique and system improve detection sensitivity over other systems employing black body radiation sources by at least 10 dB. Detection sensitivity may be improved by at least 20 dB. Detection sensitivity may be improved by at least 25 dB. Detection sensitivity may be improved up to 30 dB.

In one embodiment, the laser source may be an infrared laser source 110. One exemplary laser source employed in the application is a long wave infrared (LWIR) comb source. The LWIR comb source is well suited for high-resolution, high-sensitivity spectroscopy. The optical output from the source may be pulsed with variable repetition rates. Generally the pulse repetition rate may be greater than 1 MHz. In some embodiments, the pulse repetition rates may greater than or equal to 100 MHz. In some embodiments, the pulse repetition rates may greater than or equal to 200 MHz. The pulse repetition rate may greater than or equal to 300 MHz. The pulse repetition rate may be greater than or equal to 400 MHz. The pulse repetition rate may be less than or equal to 500 MHz. Separately, the instantaneous spectral breadth of the output pulse is greater than or equal to 100 cm⁻¹.

In an alternative embodiment, the laser source 110 may have an optical output with a pulse repetition rate exceeding 10 GHz. The high pulse repetition rate may desired based on a detector 130 that suppresses background spectral noise sources. Particularly the high pulse repetition rate may be used in combination with frequency-comb optical filters to suppress any background spectral noise sources.

Generally, the spectral breadth and pulse repetition rate are sufficiently broad to cover target spectral features of interest without additional center wavelength tuning. If surveying over a wider spectral range is required, the center wavelength of the optical output can be tuned over a broader range. The output is temporally coherent and its coherence can be utilized for rejecting the contribution from the background thermal radiation.

In one or more embodiments, the laser source 110 outputs a spatially coherent optical beams 111. Doing so enables implementation of interferometric detection techniques at the detector 130. The laser source 110 may also be temporally coherent.

The optical beam 111 is split into two beams 111a, 111b via one or more mirrors 115 upstream of the medium 120. As shown in FIG. 1, there are 2 mirrors 115 located upstream of the medium. The mirrors are configured to have different specifications to permit beam 111a to be split while beam 111b continues in substantially the same direction as optical beam 111.

As shown in FIG. 1, the medium 120 accepts beams 111a, 111b at separate entry points. Beams 111a, 111b evanescently propagate near the surface of the medium. FIG. 1 also shows beams 111a, 111b exiting the medium 120. The beams 111a, 111b leaving the medium are respectively indicated as 111a′, 111b′ since they carry spectral data. In this particular embodiment, the beams 111a′, 111b′ are interferometrically combined via one or more mirrors 125 operating as components of an interferometer prior to entering detector 130. In one embodiment, a photodetector (not shown) may be positioned proximate the mirrors of the interferometer 125. Alternatively, the photodetector may be positioned in detector 130. Functionally, the photodetector is responsible for recording modulated wavelength information of a material placed in the IR beam including an interference pattern of beams 111a′, 111b′. In other embodiments, beams 111a′, 111b′ may be directly transmitted to detector 130 for processing by an internally housed interferometer 135 (shown in dotted lines). Detection of the spectra will be discussed in further detail below.

The medium 120 is generally formed as a circular substrate. However, the substrate may be any shape as envisaged by the skilled person. More specifically, the substrate may be an attenuated total reflection (ATR) substrate crystal. The ATR substrate crystal includes a first area 120a and a second area 120b. As shown, the first and second areas 120a, 120b are separated by a barrier 125. The barrier 125 may be formed from the same material as the ATR substrate crystal. Preferably, the barrier 125 may also be formed during the manufacture of the ATR substrate crystal. Alternatively, the barrier 125 may be formed from a material other than the ATR substrate crystal.

In the embodiment shown in FIG. 1, first area 120a of the medium 120 includes a biological agent in a liquid solvent (e.g., test solution). The biological agent may be an unknown virus. The second area 120b includes a liquid solvent (e.g., reference solution). Preferably, the second area 120b is devoid of the biological agent. According to an exemplary embodiment, the liquid solvent in the first and second areas are substantially similar.

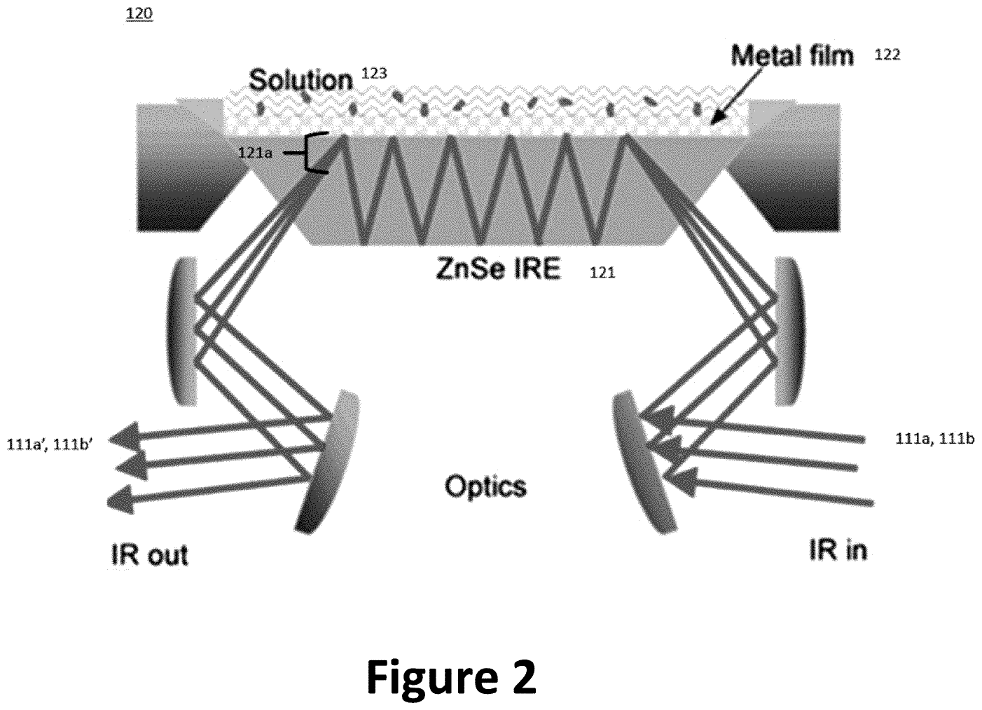

FIG. 2 illustrates a more detailed embodiment of an exemplary medium 120. The medium 120 may include multiple layers. ATR crystal substrate medium 120 and beam path in the system 100. The ATR crystal 121 is made up of multiple layers. The bottom layer 121 may be a high-index, IR transparent material. In an exemplary embodiment, the bottom layer 121 includes one or more of ZnSe, Ge, and KRS-5. The refractive index of the ATR substrate crystal 121 is higher than the IR liquid located in the second area 120 (e.g., reference path). The preferred refractive index allows for repeated total internal reflection in the ATR substrate crystal 121 caused by the optical beams 111a, 111b being transmitted into the ATR substrate crystal 121. Additionally, an evanescent IR field localized near the boundary between the ATR and test/reference solutions is achieved. Generally, the optical beams 111a, 111b, probe the medium 120 near the substrate crystal's surface 121a surface proximate to where biological agents in the test solution are located. The surface area 121 thickness is about 10-100 nm. This configuration limits the amount of solvent in the beam path.

As further shown in the medium 120 illustrated in FIG. 2, a thin film layer 122 may be deposited above the substrate crystal 121. The layer 122 may range from 10 to 100 nm in thickness. More particularly, the layer 122 may include metal nano particles to enhance the strength of the local electric field through surface plasmon enhancement. As observed by the inventors, the plasmon enhancement based upon the specific arrangement of metal nanoparticles on the substrate provides 1-3 orders of magnitude increased absorption strength. In some instances, this may be more. Additionally, a 10-100 fold increase in the absorption signal has been demonstrated using this technique. In a further exemplary embodiment, further enhancements may also be envisaged within the scope of this invention. For example, an array of nano metal antenna may also employed in layer 122 to further increase the field strength for a particular wavelength band of interest.

Next, the solution 123, including both a reference solution 123a in a first area 120a and a test solution 123b in a second area 120b, is formed on the substrate crystal 121. If a metal film layer 122 is formed on the substrate crystal 122, the solution 123 is formed on the metal film layer 122. The solvent of the solution 123 preferably is a liquid that does not have absorption features which may interfere with the target signal from the biological agent being detected and identified. Preferably, the liquid is biologically compatible with the target biological agent. One option for solvent is a biologically inert liquid. A more preferred option is a biologically inert liquid that also exhibits a reduced level of IR absorption. In an exemplary embodiment, the solvent may include Nujol and/or Flourolube.

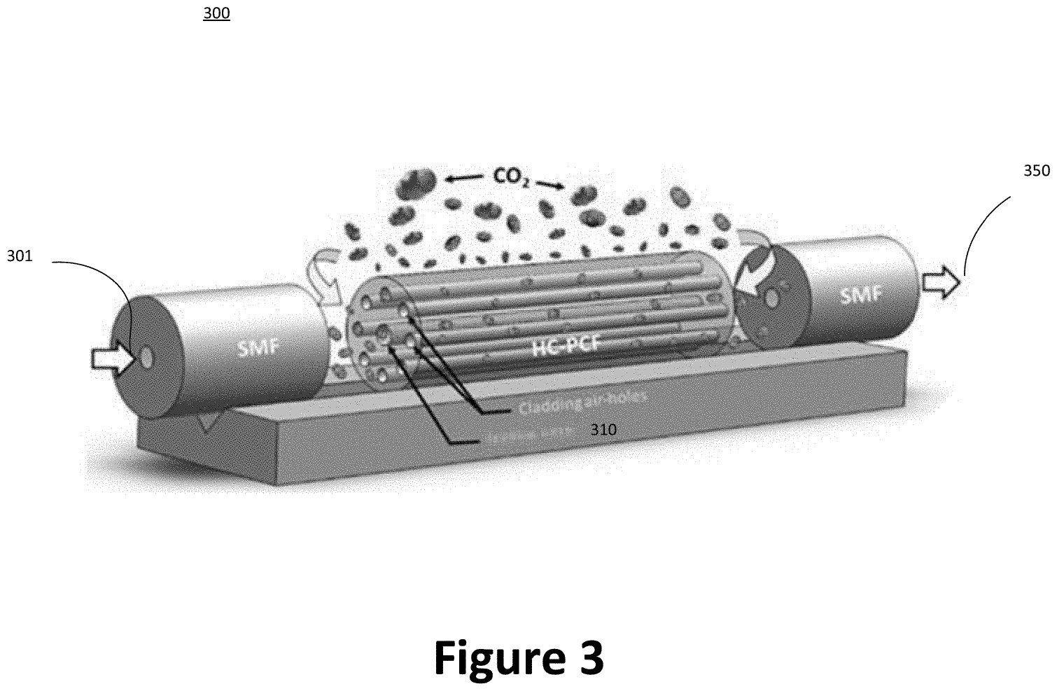

In an alternative embodiment as exemplarily shown in FIG. 3, the medium 120 may include one or more single mode fibers (SMF) each with one or more hollow-core fibers (HCFs) or capillary tubes. For purposes of this embodiment, the medium 120 will be referenced as a SMF 300. The HCF 310 may be a single mode fiber (SMF) 310 including one input 301 and one output 350 for transmitting optical beams. As determined by the inventors, HCFs increase the interaction distance between the photon and analyte. In so doing, the electric field of the photons can be improved by 1-3 orders of magnitude if not more.

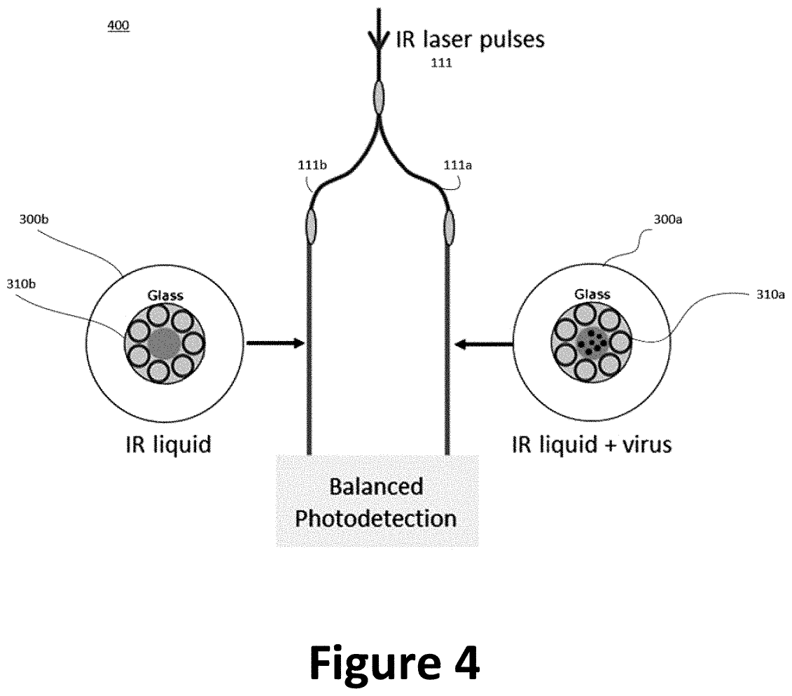

According to an exemplary embodiment, FIG. 4 shows a more detailed view of an arrangement of multiple SMFs 300a, 300b used in the system 100 shown in FIG. 1. Here, SMF 300b may include a HCF 310b. HCF 310b may be composed of glass. Additionally, HCF 310b may include the reference liquid 123b including a liquid solvent. Alternatively, SMF 300b may include HCF 310b. HCF 310b may also be composed of glass. HCF 310b may include the test liquid 123a including the biological agent in liquid solvent. FIG. 4 also illustrates a state of balanced photodetection. In this scheme, light from path 111b undergoes simple absorption/scattering due to the fiber and IR liquid, while light in path 111a undergoes absorption/scattering due to the fiber and IR liquid and analyte (i.e., virus). By subtracting these signals via balanced photodetection we can uncover the true analyte signature and reject various common mode noise effects, thereby increasing sensitivity.

Referring back to the system 100 of FIG. 1, the output beams 111a′, 111b′ from medium 120 are transmitted to an spectrometer 130, otherwise known as a spectrometer. According to an embodiment, the spectrometer 130 may be made from Ill-V semiconductors such as for example InSb, bolometers, and II-VI semiconductors such as for example Mercury-Cadmium Telluride (MCT). In an exemplary embodiment, the spectrometer 130 may include liquid-Nitrogen or thermo-electrically cooled MCT detectors used as photo-balanced receivers.

Generally, when IR radiation is passed through the medium, non-absorbed radiation in the reference and sample solutions pass through and are detected. The detector 130 may include an interferometer 135 located therein for performing interferometry (shown in dotted lines) as discussed above. A photodetector (not shown) of the interferometer 135 measures intensity of transmitted or reflected light as a function of its wavelength to collect interference pattern information, i.e., interferogram, of the object being studied. Background subtraction of the common mode may be performed electronically. More particularly, achievable extinction (or common-mode rejection) at the interferometer depends upon a few factors. These factors at least include: balancing of the optical powers in the two distinct beam paths described above, their spatial beam coherence and overlap, and the optical bandwidth of the optics in the interferometer. It is envisaged that better than 20 dB common mode rejection can be achieved.

In an exemplary embodiment, the spectrometer 130 may be a Fourier Transform Infrared spectrometer (FTIR). A Fourier Transform algorithm employed on a computing machine may identify and/or quantify the material based on plots of intensity versus wavenumbers representing a molecular ‘fingerprint’ of the sample.

When compared to black body radiation sources made of a ceramic, the combination of synchronous detection and interferometry with a pulsed optical beam of high frequency can lead to a signal-to-noise ratio (SNR) improvements up to about 30 dB. This is due to the benefit of lock-in detection, where the signal of interest is shifted to a high frequency away from DC. Since most noise sources have a frequency spectrum centered close to DC, much of the system noise is avoided using this technique. The SNR improvement may preferably be up to about 40 dB. The SNR improvement may be up to about 50 dB. The improved SNR may offer detection of up to 10 picograms of analytes. This amount of analyte is particularly important for detecting biological agents such as the coronavirus, its variants, or other similarly sized viruses. Nanograms of the target virus may induce approximately 10⁻⁵ relative change in the optical spectrum of the interrogating infrared pulses. While detection of these minute changes in the spectrum is challenging due to the limited dynamic ranges of the detector and signal processing electronics, the above-mentioned technique for interferometric subtraction of the common mode helps

Moreover, in view of the exemplary arrangement of the system 100 including the laser source 110 configured to deliver pulsed optical beams and the exemplary spectrometer 130 described above, the 1/f noise of detectors and other lower frequency noises can be discriminated. That is, the lower frequency noises are immaterial in view of the high frequencies employed at pulse repetition rates exceeding 100 MHz.

### Machine Learning Model

According to even another aspect of the application, artificial intelligence such as machine learning models, are employed to improve identification of a virus. Ultimately, a judicious use of solvent and a signal-boosting measurement configuration in combination with a pattern recognition algorithm will improve identification of the target virus in the presence of other biochemical interferers.

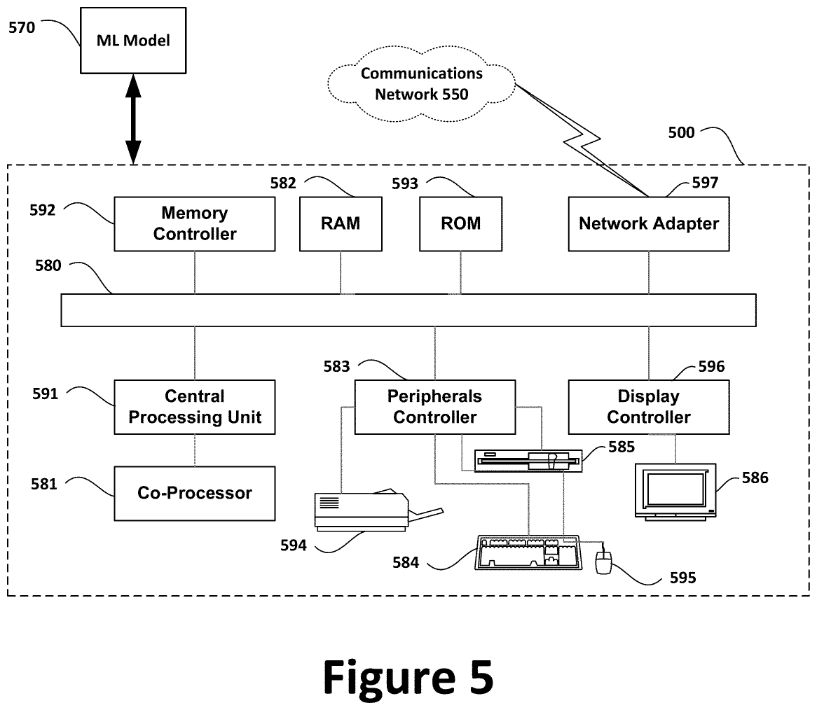

FIG. 5 illustrates a block diagram of an exemplary computing system 500 which may be used with the system 100. The computing system 500 may comprise a computer or server and may be controlled primarily by computer readable instructions (e.g., stored on a non-transitory computer-readable medium), which may be in the form of software, wherever, or by whatever means such software is stored or accessed. Such computer readable instructions may be executed within a processor, such as central processing unit (CPU) 591, to cause computing system 500 to do work. In many known workstations, servers, and personal computers, central processing unit 591 is implemented by a single-chip CPU called a microprocessor. In other machines, the central processing unit 591 may comprise multiple processors. Coprocessor 581 is an optional processor, distinct from main CPU 591, that performs additional functions or assists CPU 591.

In operation, CPU 591 fetches, decodes, and executes instructions, and transfers information to and from other resources via the computer's main data-transfer path, system bus 580. Such a system bus connects the components in computing system 500 and defines the medium for data exchange. System bus 580 typically includes data lines for sending data, address lines for sending addresses, and control lines for sending interrupts and for operating the system bus. An example of such a system bus 580 is the PCI (Peripheral Component Interconnect) bus.

Memories coupled to system bus 580 include random access memory (RAM) 82 and read only memory (ROM) 593. Such memories include circuitry that allows information to be stored and retrieved. ROMs 593 generally contain stored data that cannot easily be modified. Data stored in RAM 582 may be read or changed by CPU 91 or other hardware devices. Access to RAM 582 and/or ROM 593 may be controlled by memory controller 92. Memory controller 92 may provide an address translation function that translates virtual addresses into physical addresses as instructions are executed. Memory controller 592 may also provide a memory protection function that isolates processes within the system and isolates system processes from user processes. Thus, a program running in a first mode may access only memory mapped by its own process virtual address space; it cannot access memory within another process's virtual address space unless memory sharing between the processes has been set up.

In addition, computing system 500 may contain peripherals controller 583 responsible for communicating instructions from CPU 591 to peripherals, such as printer 594, keyboard 584, mouse 595, and disk drive 585.

Display 586, which is controlled by display controller 596, is used to display visual output generated by computing system 500. Such visual output may include text, graphics, animated graphics, and video. Display 586 may be implemented with a CRT-based video display, an LCD-based flat-panel display, gas plasma-based flat-panel display, or a touch-panel. Display controller 96 includes electronic components required to generate a video signal that is sent to display 86.

Further, computing system 500 may contain communication circuitry, such as for example a network adaptor 597, that may be used to connect computing system 500 to an external communications network, such as network 550, to enable the computing system 500 to communicate with other computing systems in the network.

Further, the processor 91 and/or co-processor 81 of the computing system 500 may be configured to execute machine-readable computer program instructions stored in the RAM 82. The computer program instructions may be based upon one or more of an information component, a training component, a prediction component and/or other components.

The computing system 500 may also include a machine learning model 570 as shown in FIG. 5. The machine learning model 570 may also be operably coupled to the computing system.

As shown by the bi-directional arrows indicative of data transfer and sharing. The ML model 570 may receive information collected from the spectrometer 130 regarding specific biological agents. The information may be used to detect variants or mutations.

ML model 570 may be built from scratch or be built by further training an existing ML model. The ML model 570 may also be trained on an ongoing basis, as indicated by the respective arrows 640a-c. Specifically, the ML model 570 may be used by the computing system to [What is the ML model checking if the FTIR identifies the biological agent?]

Artificial neural networks (ANNs) are models used in machine learning and may include statistical learning algorithms conceived from biological neural networks (particularly of the brain in the central nervous system of an animal) in machine learning and cognitive science. ANNs may refer generally to models that have artificial neurons (nodes) forming a network through synaptic interconnections (weights), and acquires problem-solving capability as the strengths of the interconnections are adjusted, e.g., at least throughout training. The terms ‘artificial neural network’ and ‘neural network’ may be used interchangeably herein.

Disclosed implementations of artificial neural networks may apply a weight and transform the input data by applying a function, this transformation being a neural layer. The function may be linear or, more preferably, a nonlinear activation function, such as a logistic sigmoid, Tan h, or rectified linear activation function (ReLU) function. Intermediate outputs of one layer may be used as the input into a next layer. The neural network through repeated transformations learns multiple layers that may be combined into a final layer that makes predictions. This learning (i.e., training) may be performed by varying weights or parameters to minimize the difference between the predictions and expected values. In some embodiments, information may be fed forward from one layer to the next. In these or other embodiments, the neural network may have memory or feedback loops that form, e.g., a neural network. Some embodiments may cause parameters to be adjusted, e.g., via back-propagation.

An ANN is characterized by features of its model, the features including an activation function, a loss or cost function, a learning algorithm, an optimization algorithm, and so forth. The structure of an ANN may be determined by a number of factors, including the number of hidden layers, the number of hidden nodes included in each hidden layer, input feature vectors, target feature vectors, and so forth. Hyperparameters may include various parameters which need to be initially set for learning, much like the initial values of model parameters. The model parameters may include various parameters sought to be determined through learning. And the hyperparameters are set before learning, and model parameters can be set through learning to specify the architecture of the ANN.

Learning rate and accuracy of an ANN rely not only on the structure and learning optimization algorithms of the ANN but also on the hyperparameters thereof. Therefore, in order to obtain a good learning model, it is important to choose a proper structure and learning algorithms for the ANN, but also to choose proper hyperparameters.

The hyperparameters may include initial values of weights and biases between nodes, mini-batch size, iteration number, learning rate, and so forth. Furthermore, the model parameters may include a weight between nodes, a bias between nodes, and so forth.

In general, the ANN is first trained by experimentally setting hyperparameters to various values, and based on the results of training, the hyperparameters can be set to optimal values that provide a stable learning rate and accuracy.

In some embodiments, the learning of models may be supervised, and/or unsupervised type. For example, there may be a model for certain predictions that is learned with one of these types but another model for other predictions may be learned with another of these types.

Supervised learning is the machine learning task of learning a function that maps an input to an output based on example input-output pairs. It may infer a function from labeled training data comprising a set of training examples. In supervised learning, each example is a pair consisting of an input object (typically a vector) and a desired output value (the supervisory signal). A supervised learning algorithm analyzes the training data and produces an inferred function, which can be used for mapping new examples. And the algorithm may correctly determine the class labels for unseen instances.

Unsupervised learning is a type of machine learning that looks for previously undetected patterns in a dataset with no pre-existing labels. In contrast to supervised learning that usually makes use of human-labeled data, unsupervised learning does not via principal component (e.g., to preprocess and reduce the dimensionality of high-dimensional datasets while preserving the original structure and relationships inherent to the original dataset) and cluster analysis (e.g., which identifies commonalities in the data and reacts based on the presence or absence of such commonalities in each new piece of data). Semi-supervised learning is also contemplated, which makes use of supervised and unsupervised techniques.

The ML model 570 may analyze and make predictions against a reference set of data called the validation set. In some use cases, the reference outputs may be provided as input to the prediction models, which the prediction model may utilize to determine whether its predictions are accurate, to determine the level of accuracy or completeness with respect to the validation set data, or to make other determinations. Such determinations may be utilized by the prediction models to improve the accuracy or completeness of their predictions. In another use case, accuracy or completeness indications with respect to the prediction models' predictions may be provided to the prediction model, which, in turn, may utilize the accuracy or completeness indications to improve the accuracy or completeness of its predictions with respect to input data. For example, a labeled training dataset may enable model improvement. That is, the training model may use a validation set of data to iterate over model parameters until the point where it arrives at a final set of parameters/weights to use in the model.

Training data may be obtained from the RAM 583 of FIG. 5 which may comprise hundreds, thousands, or even many millions of pieces of information (e.g., images or other sensed data). The dataset may be split between training, validation, and test sets in any suitable fashion. For example, some embodiments may use about 60% or 80% of the images for training or validation, and the other about 40% or 20% may be used for validation or testing. In another example, the labelled images may be randomly split, the exact ratio of training versus test data varying throughout. When a satisfactory model is found, prediction component 34 may train it on 95% of the training data and validate it further on the remaining 5%.

The validation set may also be a subset of the training data, which is kept hidden from the model to test accuracy of the model. The test set may be a dataset, which is new to the model to test accuracy of the model. The training dataset used to train prediction models 64 may leverage, via prediction component 34, an SQL server and a Pivotal Greenplum database for data storage and extraction purposes.

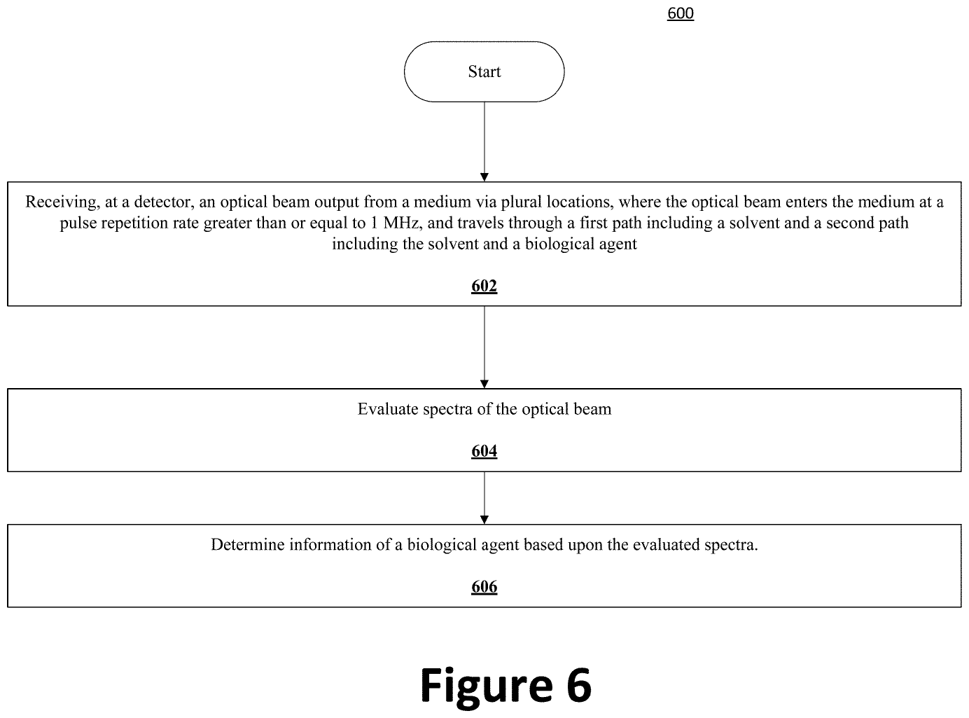

According to another aspect as shown in FIG. 6, illustrates an exemplary method 600 of the present application. Step 602 describes receiving, at a detector, an optical beam output from a medium via plural locations, where the optical beam enters the medium at a pulse repetition rate greater than or equal to 100 MHz. the pulsed optical beam travels through a first path including a solvent and a second path including the solvent and a biological agent. In Step 604, spectra of the optical beam leaving the medium is evaluated. This may done by the spectrometer, preferably a FTIR spectrometer. In Step 606, information of a biological agent is determined in view of the evaluated spectra.

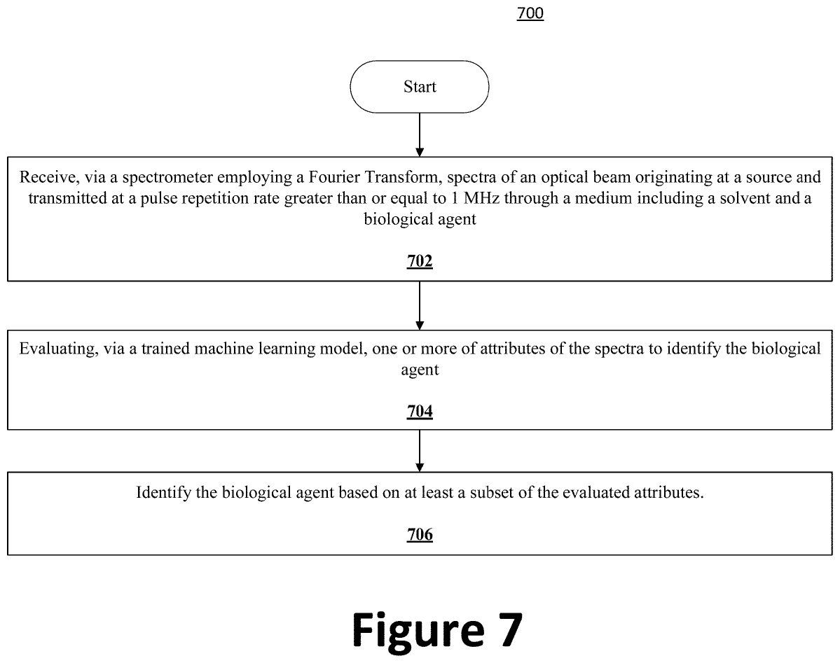

According to yet another aspect as illustrated in FIG. 7, a computer readable medium including program instructions 700 is described. The program instructions are executable by a processor of a computing device. Upon execution by the processor, the computing device is caused to receive, via a spectrometer employing a Fourier Transform, spectra of an optical beam originating at a source. The optical beam is transmitted at a pulse repetition rate greater than or equal to 1 MHz through a medium including a solvent and a biological agent (702). It is envisaged the pulse repetition rate may be greater than 1 MHz. The pulse repetition rate may be greater than 100 MHz Upon execution by the processor, the computing device is also caused to evaluate, via a trained ML model, one or more attributes of the spectra to identify the particular biological agent. In a preferred embodiment, the biological agent is a virus (704). The ML model is described above in FIG. 5. The ML model 570 may reside within the computing device 500. Alternatively, the ML model 570 may operably communicate with the computing device 500 via communication network 550. Subsequently, upon execution by the processor, the computing device is caused to identify the biological agent based on at least one of the evaluated attributes (706).

In a particular use case of the present application, the system may be used in the field. More particularly, the system may be positioned at a drive-through testing center. The testing center is configured to have capabilities to swab a patient. The swabbed sample can be tested directly by the above-mentioned systems described above. Within a matter of minutes, the clinician may receive an output from the system regarding a particular biological agent being examined. In a preferred embodiment, it may be attributed to the COVID-19 virus.

As shown in FIG. 7, the system may employ ML models to improve throughput and accuracy. This is achieved by examining one or more attributes of the spectra. The more attributes which are examining may result in better accuracy. The present application envisages modifying the number of attributes to examine to meet particular time constraints. With the ML model 570 continuously learning with more testing data, its speed and reliability will improve.

While the system and method have been described in terms of what are presently considered to be specific embodiments, the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1. A system comprising:
an infrared optical source configured to output an optical beam at a pulse repetition rate greater than or equal to 1 MHz;
a medium configured to receive the optical beam in first and second locations of the medium, where each of the first and second locations is separated by a barrier, the first location incudes a solvent, and the second location includes a biological agent in the solvent; and
a detector configured to receive the outputted optical beam from the medium and detect infrared spectra therefrom.

2. The system of claim 1, further comprising a means for interferometrically subtracting a common mode of the received optical beam prior to the detection of infrared spectra.

3. The system of claim 2, wherein a rejection of the common mode ranges between about 20-30 dB.

4. The system of claim 3, exhibiting an improvement in a signal-to-noise ratio up to about 50 dB employing the infrared optical source in comparison to another system employing a black body radiation source.

5. The system of claim 1, wherein the pulse repetition rate is less than about 500 MHz.

6. The system of claim 1, wherein the optical beam is spatially coherent.

7. The system of claim 1, wherein the medium comprises a crystalline substrate and a metal film layer of about 10 to 100 nm thickness formed on the crystalline substrate.

8. The system of claim 1, wherein the medium includes plural hollow core fibers.

9. The system of claim 1, wherein the pulse repetition rate is greater than or equal to 10 GHz.

10. A method comprising:
receiving, at a detector, an optical beam output from a medium via plural locations, where the optical beam enters the medium at a pulse repetition rate greater than or equal to 1 MHz, and travels through a first path including a solvent and a second path including the solvent and a biological agent;
evaluating spectra of the optical beam; and
determining information of a biological agent based upon the evaluated spectra.

11. The method of claim 10, further comprising:
interferometrically subtracting a common mode of the received optical beam prior to the evaluation of spectra.

12. The method of claim 11, wherein a rejection of the common mode is greater than about 20 dB.

13. The method of claim 10, further comprising:
detecting about 1-10 picograms of analytes from the biological agent.

14. A computer readable medium including program instructions that, when executed by a processor of a computing device, causes the computing device to:
receive, via a spectrometer employing a Fourier Transform, spectra of an optical beam originating at a source and transmitted at a pulse repetition rate greater than or equal to 1 MHz through a medium including a solvent and a biological agent;
evaluate, via a trained machine learning model, one or more of attributes of the spectra to identify the biological agent; and
identify the biological agent based on at least a subset of the evaluated attributes.

15. The computer readable medium of claim 14, wherein the evaluation includes assigning each of the attributes a score based upon a likelihood of similarity with at least one respective attribute in training data used by the machine learning model.

16. The computer readable medium of claim 14, wherein the program instructions when executed by the processor further causes the computing device to transmit the identified biological agent over a network to another computing device.

17. The computer readable medium of claim 14, wherein interferometric subtraction of a common mode of the optical beam occurs prior to receiving the spectra.

18. The computer readable medium of claim 17, wherein a rejection of the common mode is greater than about 20 dB.

19. The computer readable medium of claim 14, wherein about 1-10 picograms of analytes of the biological agent are captured from the optical beam to produce the spectra.

20. The computer readable medium of claim 14, wherein the pulse repetition rate is less than about 500 MHz.
